(19) United States
(12) Patent Application Publication
(10) Pub. No.: US 2011/0172552 A1
(43) Pub. Date: Jul. 14, 2011

(54) ACOUSTIC SLEEP APNEA MONITOR

(75) Inventors: Brian S. Rothman, Franklin, TN (US); James M. Berry, Nashville, TN (US)

(73) Assignee: VANDERBILT UNIVERSITY, Nashville, TN (US)

(21) Appl. No.: 12/985,490

(22) Filed: Jan. 6, 2011

Related U.S. Application Data
(60) Provisional application No. 61/294,244, filed on Jan. 12, 2010.

(51) Int. Cl. A61B 5/08

(52) U.S. Cl. 600/529

(57) ABSTRACT

The present invention discloses an apparatus and method for monitoring an individual for an irregular respiratory event. The present invention discloses a sleep apnea monitor for monitoring an individual for an apneic event. The monitor includes a microphone for detecting tracheal sounds related to respiration so that irregular lengths of time between such sounds may activate an alert of the medical condition. The method of monitoring an individual for an irregular respiratory event includes attaching the monitor in a nonintrusive manner to a location around the throat area.

This application claims the benefit of U.S. Provisional Patent Application Ser. No. 61/294,244, filed Jan. 12, 2010, entitled “Acoustic Sleep Apnea Monitor” which is hereby incorporated by reference in its entirety.

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

Obstructive sleep apnea (OSA) affects up to 18 million Americans with an estimated 10 million Americans who are not diagnosed. OSA is a prevalent condition that restricts breathing while a person is sleeping. Individuals with OSA tend to be more sensitive to otherwise non-obstructive doses of sedatives which relax the musculature of the pharynx and throat causing obstruction. OSA causes cerebral hypoxia and reduces the memory capacity of the brain. Sedative administration to patients who have apneic events can be put them at risk for life-threatening apnea. Continuous monitoring of at-risk patients currently requires telemetry which is an expensive and limited resource. OSA can make intubation difficult and advanced knowledge of the condition is useful if a secure airway is necessary. In addition to OSA, there are other respiratory events that are monitored with intrusive and expensive equipment. What is needed is an inexpensive, easy to use, and reliable way to monitor for apneic events and other respiratory events.

### SUMMARY OF INVENTION

Disclosed herein is sleep apnea monitor requiring only nonintrusive attachment to the skin of the throat area of a subject, and being so low in cost that the monitor is disposable. As further described herein, the present invention is a monitor having an alarm system which is activated during an apnea event, or other abnormal respiratory event. The monitor includes audible, visual, and sensory alarms. The invention disclosed herein is particularly relevant to individuals having compromised respiratory function, such as postoperative patients, or other individuals that are receiving sedatives. The present invention may be attached to the throat area by use of a medical adhesive. After it is in contact with the throat area, it is ready to use, as it is a self-contained and self-energized device. The monitor may be used repeatedly as it's power/reset button may be used to reset the monitor after an alert is triggered due to an irregular respiratory event.

Disclosed herein is an apnea monitor, including, a housing, a microphone attached to the housing, an amplifier attached to the microphone, the amplifier having a bandpass filter, a microprocessor attached to the amplifier, a reset button attached to the microprocessor, a light attached to the microprocessor, an adhesive arm attached to the housing, and an energy source attached to the microprocessor. In certain embodiments of the invention, the apnea monitor further includes a speaker attached to the microprocessor. In still other embodiments, the apnea monitor weighs thirty grams or less. In yet other embodiments, the bandpass filter accepts frequencies in a range of from about 1.5 kHz to about 2.0 kHz, or in a range of from about 400 Hz to about 700 Hz.

In still other embodiments, the apnea monitor includes, an adhesive element, a microphone attached to the adhesive element, a microprocessor attached to the microphone, a light attached to the microprocessor, a speaker attached to the microprocessor and a battery attached to the microprocessor. In still other embodiments, the apnea monitor is disposable. In yet other embodiments, the light illuminates upon the detection of an apneic event. In other embodiments of the present invention, the apnea monitor further includes a vibration unit attached to the microprocessor. In still other embodiments, the adhesive element is circular. In yet other embodiments, the microprocessor further includes software for audio analysis. In certain embodiments, the apnea monitor further includes a reset button attached to the microprocessor. In still other embodiments, the apnea monitor further includes a light source to indicate power. In yet other embodiments, the microprocessor further comprises wireless communication hardware.

Accordingly, one object of the present invention is to provide an apparatus for use in monitoring a subject for an apneic event.

Another object of the present invention is to provide an apparatus that is disposable, and easily attached to an subject in order to monitor that subject for an apneic event.

Still another object of the present invention is to provide a method of monitoring a subject for an irregular respiratory event.

Still another object of the present invention is to provide an apparatus for nonintrusive and inexpensive monitoring of a subject for an irregular respiratory event.

### BRIEF DESCRIPTION OF THE DRAWINGS

FIG. 1 is a side view of an embodiment of the monitor disclosed herein. Shown therein is the housing, adhesive pieces extending laterally, and contact microphone. The broken line depicts the surface of the skin of a subject.

FIG. 2 is a bottom view of an embodiment of the monitor disclosed herein. Shown there is the contact surface of the housing, a circular adhesive piece, and the contact microphone.

FIG. 3 is a top view of an embodiment of the monitor disclosed herein. Shown there is the housing of the monitor, power/reset button, LED light, and speaker.

FIG. 4 is a schematic diagram of the electrical and communication connections of an embodiment of the invention disclosed herein. Shown therein is an embodiment of the monitor, having contact microphone, amplifier with bandpass filter, microprocessor, energy source, speaker, LED light, and power/reset button.

FIG. 5 is a schematic diagram of the electrical and communication connections of an embodiment of the invention disclosed herein. Shown therein is an embodiment of the monitor, having contact microphone connected directly to the microprocessor, as well as the other connections shown.

### DESCRIPTION OF THE PREFERRED EMBODIMENTS

The present invention is an acoustic sleep apnea monitor. In certain embodiments, the invention, referred to as a monitor 10, includes a contact microphone 12, an amplifier 14 having a bandpass filter, a microprocessor 16, an energy source 18, a light 20, a speaker 22, and adhesive piece 24. The monitor 10 may be placed on a person so that the contact microphone 12 is in contact with the pretracheal skin. That will allow the contact microphone 12 to detect tracheal sounds. In certain embodiments of the present invention, detection of such tracheal sounds resets a countdown timer such that if a tracheal sound is not detected within a given period of time, an alarm is triggered. The alarm may be either visual, auditory, sensory, or any combination thereof. The monitor 10 is highly desirable as it is inexpensive, small, lightweight, and may be used as a medical disposable. Further, the present invention differs from the telemetry systems in use today which measure actual airflow, pressure changes, or carbon dioxide return. The present invention measures tracheal sounds. The present invention allows for the monitoring of postoperative patients who have reduced hypoxic drive and an increased incidence of apnea. Further, the present invention may be used to monitor apneic events when sedatives are given, so that expensive telemetry is not needed where hypoxia is a late sign of apnea.

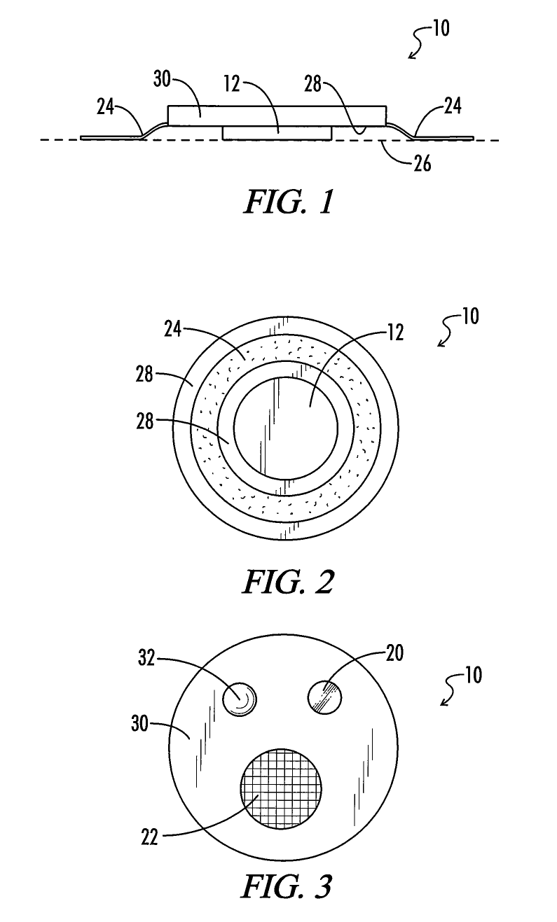

Referring now to FIG. 1, there is shown a side view of the monitor 10 in position to detect trachea sounds of a person 26. The monitor 10 adheres to the person 26 by use of adhesive pieces 24. In certain embodiments, the adhesive pieces 24 may contact and attach to the skin of the person 26 at a location that is lateral to the monitor 10, as shown in FIG. 1. The embodiment shown in FIG. 1 shows the adhesive piece 24 extending laterally from the monitor 10. Accordingly, the adhesive piece 24 are attached to the housing 30 of the monitor 10. In other embodiments, the adhesive piece 24 may be attached to the contact surface 28 of the monitor 10, as best seen in FIG. 2. In certain embodiments, the monitor 10 may be attached above the suprasternal notch. The adhesive piece 24 may be an element with an adhesive material which readily sticks to and adheres to a surface such as human skin. In alternate embodiments, the adhesive piece 24 may provide sufficient contact between the contact microphone 12 and the general neck area so that tracheal sounds are detected and the monitor 10 is in a fixed position for a sufficient period of time, as known to those of skill in the art. The function of the adhesive piece 24 is to attach the monitor 10 to the skin of the person 26. Such adhesives are well known and readily commercially available. An example of such an adhesive is a precordial adhesive disk, or a product named Tegaderm, which is commercially available from 3M of St. Paul, Minn. Such an embodiment is shown in FIG. 2, which shows the monitor 10 from the bottom side.

Referring now to FIG. 3, there is shown a top view of the present invention. As shown, the monitor 10 includes a housing 30, speaker 22, a reset button 32 and a light 20. As used herein, a reset button 32 may mean a reset switch, power switch, or on/off switch. Such switches are well known and are readily commercially available. In certain embodiments, the reset button 32 may also include a light source to indicate power. Reset buttons are well know in the art and are readily commercially available. In other embodiments, the light 20 may be a light emitting diode (LED) which functions as a visual alarm such that it lights up, or blinks, when an alert is signaled by the monitor 10. Light sources, such as LED, and other suitable light sources, are well known in the art and are readily commercially available. The light 20 is a component of the present invention which is capable of displaying visible light. In certain embodiments of the present invention, the light 20 is a liquid crystal display (LCD). The light 20 does not generate sufficient heat, or produce any byproducts, chemical or otherwise, that are harmful to the surface of the subject. In certain embodiments, the speaker 22 may be activated upon the monitor 10 providing an alert. The speaker 22 should be sufficient to provide an audible signal which could be detected by a person near the monitor 10. Speakers having suitable size and sound characteristics are well know in the art and readily commercially available. Regarding the specific positions of the reset button 32, light 20, and speaker 22, they may be positioned as desired on the top surface of the monitor 10 or on any side surface. The housing 30 of the monitor 10 may be any suitable lightweight, biocompatible material such as, an appropriate plastic, rubber, or metal, or the like. Methods of manufacturing and shaping materials suitable for the housing 30 are known to those skilled in the art and such services are readily commercially available. In certain embodiments of the present invention, the size of the monitor 10 is approximately two centimeters in diameter with a thickness of less than two centimeters. In other embodiments of the present invention, the monitor 10 may have an alternate shape, such as a square, rectangle, or the like. In such embodiments, the monitor 10 has dimensions of two centimeters in length by two centimeters in height. In still other embodiments the monitor 10 may have the dimensions disclosed herein or smaller. In certain embodiments of the present invention, the monitor 10 weighs thirty grams or less.

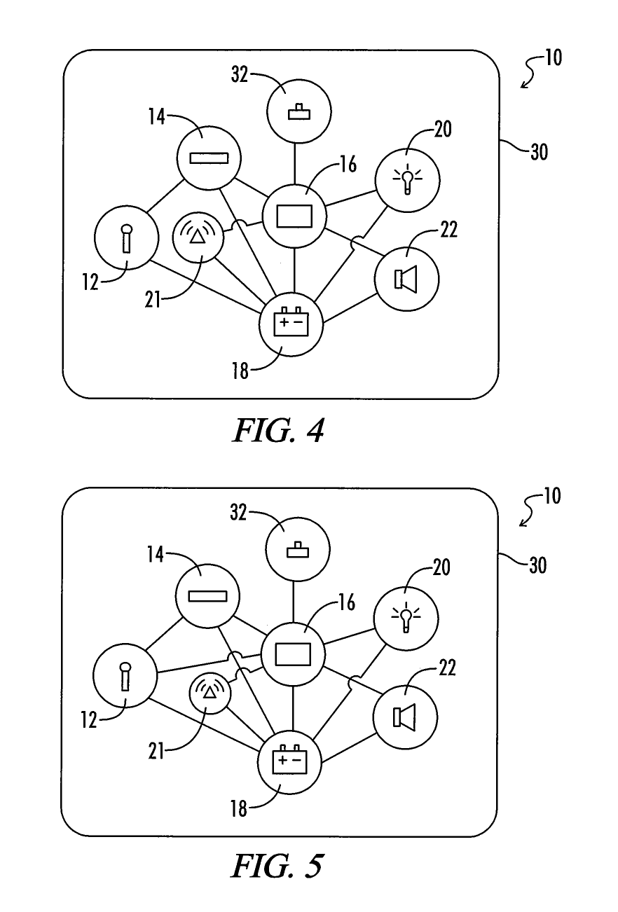

Referring now to FIG. 4, there is shown a schematic diagram of the monitor 10. Shown therein is an energy source 18 operationally connected to the microprocessor 16. Note that the connections between the components of the present invention are those operational connections known to those of ordinary skill in the arts. The schematic diagram uses lines to demonstrate the operational connectivity of the parts shown (i.e., wires, or other means, attaching, or allowing communication, or connectivity, so that, for example, the microprocessor 16 signals an alert such that the speaker 22 sounds, the light 20 blinks, or the like). Operational connectivity includes any connections necessary for power, data or information transfer, or the like, for the operation of the specific device. One of ordinary skill in the art is familiar with such types of connections. For example, as seen in FIG. 4, the reset button 32 is operationally connected to the microprocessor 16. As a second example, the microprocessor 16 is operationally connected to the speaker 22, the light 20, the amplifier 14 having a bandpass filter, and the energy source 18.

In certain other embodiments of the present invention, the contact microphone 12 and speaker 22 may be provided by modifying a piezo transducer. The resulting contact microphone 12 is then operationally connected to the microprocessor 16, not to an amplifier 14, as best seen in FIG. 5. The resulting speaker 22 is operationally connected to the microprocessor 16 as an output device, for an alert. Piezo transducers are readily commercially available. For example, a piezo transducer is commercially available from Radio Shack Corporation of Fort Worth, Tex. as part number 273-073. In certain embodiments, modifying the piezo transducer is known to one of skill in the art and includes the steps of removing the transducer from its plastic casing and splicing an audio cable to the transducer in order to create an operational connection. Briefly, splicing includes the steps of cutting an audio cable and twisting together the exterior wires to be separate from the interior wires. Connect the two piezo transducer wires to the two audio cable wires, as known to those of skill in the art. Place an insulating material around the outside of each connection, as known to those of skill in the art. Other contact microphones 12 and speakers 22 are readily commercially available. In still other embodiments of the present invention, the microphone 12 may be an electret microphone, such as model no. 270-092 which is commercially available from Radio Shack Corporation of Fort Worth, Tex. Such microphone may be attached to a stethoscope (model no. DS-9291 from Primacare) in order to amplify breath sounds. In certain embodiments, the stethoscope amplification chamber and diaphragm were separated. Then a small washer and the microphone 12 were attached with epoxy (JB Kwik epoxy). The leads of the microphone were soldered to a 6 foot shielded cable with ⅛ inch audio jack (model no. 42-2434 from Radio Shack).

In certain embodiments of the present invention, a suitable amplifier 14 having an analog bandpass filter has the characteristics identifying and detecting the optimal frequency of breath sounds of a subject, and as described herein. Specifically, the analog bandpass filter includes resistors, capacitors, inductors and op-amps, as known to those of skill in the art. The analog bandpass filter, and parts thereof, including resistors, capacitors, inductors and op-amps are well known and readily commercially available, for example from Radio Shack Corporation of Fort Worth, Tex. A bandpass filter for the desired sound frequencies, as disclosed herein, filters the sound detected by the contact microphone 12 so that the specific sound frequencies are then provided to the microprocessor 16. In other embodiments, suitable amplifiers and filters may be used.

In certain embodiments of the present invention, the microprocessor 16 includes an analog to digital converter. Such converters are readily commercially available. Examples of such digital signal processors and microprocessors include Texas Instruments Incorporated of Dallas, Tex. or Analog Devices, Inc. of Norwood, Mass. Various software for audio recording, manipulation, and analysis may be used in conjunction with the present invention. By way of illustration, and not limitation, such software includes software available at www.sourceforge.net, LabView software which is commercially available from National Instruments, of Austin, Tex., and Matlab software which is commercially available from The Mathworks, Inc. of Natick, Mass. In certain embodiments, such software uses the Fast Fourier Transform (FFT) algorithm to calculate the discrete Fourier Transform (DFT), as known to those of ordinary skill in the art.

Upon detection of an irregular respiratory event, as further described herein, in certain embodiments of the present invention, the alert may include individual or multiple audible alarms, such as speakers 22, or visual alarms such as an individual light or multiple lights. Other embodiments may include a sensory alert, such as a vibration unit 21. Vibration units 21 are well known in the art and are readily commercially available. In still other embodiments of the present invention, an alert may include a wireless communication to another device, such as a computer, or other surveillance equipment used in connection with patient care. Such wireless communication hardware is known to those of skill in the art and is readily commercially available.

The present invention may use various power sources and power supplies as described herein, or known to those of ordinary skill in the arts. In certain embodiments, the energy source 18 is attached to, and provides a power source for, the elements disclosed herein needing power for operation. In certain embodiments, the energy source 18 may be a watch battery, such as a lithium-ion battery, or the like. Such batteries are known in the art and are readily commercially available. In certain embodiments of the present invention, the energy source 18 is removable battery. In other embodiments, the energy source 18 may be any energy source known by those of ordinary skill in the art which would provide sufficient power to the other elements for their operation in the manner described herein. In still other embodiments, the energy source 18 is a non-removable battery. In certain embodiments, the invention may include a resistor in order to match the electrical capabilities of the energy source 18 with the output ability of the other elements described herein. The present invention includes proper electrical insulation, as known by those skilled in the art, so that a subject is not shocked and so that proper function occurs under the use circumstances described herein.

Still referring to FIG. 4, there is shown an embodiment of the connection of the elements of the monitor 10. In another embodiment of the present invention, the contact microphone 12 and the speaker 22 may be a single unit, which is readily commercially available. Those elements shown in FIG. 4 are known to those of skill in the art and are readily commercially available. By way of example, and not limitation, in certain embodiments, the contact microphone 12 is commercially available from Radio Shack Corporation of Fort Worth, Tex. The microprocessor 16 may be a computer, controller, microprocessor, or processor that can receive the detected audible signal and compare that to known settings, as further disclosed herein, in order to signal an alert. Such microprocessors 16 are known to those of skill in the art and are readily commercially available, for example from Texas Instruments Incorporated of Dallas, Tex. or Analog Devices, Inc. of Norwood, Mass. The reset button 32, speaker 22 and light 20 are widely commercially available.

The detection of tracheal sounds by a contact microphone 12 results in the need to filer those sounds in order to determine whether respirator related sounds are present. Accordingly, the present invention discloses an amplifier 14 having a bandpass filter. Such an amplifier 14 first amplifies the tracheal sounds and then accepts frequencies within a certain range and rejects frequencies outside that range. In certain embodiments of the present invention, the range of sound frequencies is from about 1.5 kHz (kilohertz) to about 2.0 kHz. In other embodiments, the range of sound frequencies is representative of an obstructed airway. Such sound frequencies are known in the art. In still other embodiments, the range of sound frequencies is representative of a partially obstructed airway. Such sound frequencies are known in the art. Golabbakhsh, M., and Z. Moussavi, “Relationship Between Airflow and Frequency-Based Features Tracheal Respiratory Sound,” Electrical and Computer Engineering 2 (2004): 751-754, which is hereby incorporated by reference in its entirety. In still other embodiments of the present invention, the amplifier 14 accepts frequencies within a range of from about 400 Hz to about 700 Hz.

For certain embodiments of the present invention, the decision pathway leading to an alert is disclosed herein. The microprocessor 16 receives sound frequencies for a given range from the amplifier 14 having a bandpass filter. When such sound frequencies are received, a timed period of 15 seconds initiates. If such sound frequencies are not again received within the 15 second period, then an alert results. In other embodiments, the time period may be from about 10 seconds to about 30 seconds. In still other embodiments, the time period may be from about 15 seconds to about 20 seconds. In other embodiments, an alert results when sound frequencies within the given range occur at a rate of more than 30 respirations per minute or another rate to indicate subject distress. In still other embodiments, an alert results when sound frequencies within the given range occur at a rate of 15 or more per hour.

This patent application expressly incorporates by reference all patents, references, and publications disclosed herein.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. An apnea monitor, comprising:
a housing;
a microphone attached to the housing;
an amplifier attached to the microphone, wherein the amplifier has a bandpass filter;
a microprocessor attached to the amplifier;
a reset button attached to the microprocessor;
a light attached to the microprocessor;
an adhesive element attached to the housing;
an energy source attached to the microprocessor.

2. The apnea monitor of claim 1, further comprising a speaker attached to the microprocessor.

3. The apnea monitor of claim 2, wherein the apnea monitor weighs thirty grams or less.

4. The apnea monitor of claim 2, wherein the bandpass filter accepts frequencies in a range of from about 1.5 kHz to about 2.0 kHz.

5. The apnea monitor of claim 2, wherein the bandpass filter accepts frequencies in a range of from about 400 Hz to about 700 Hz.

6. An apnea monitor, comprising:
an adhesive element
a microphone attached to the adhesive element;
a microprocessor attached to the microphone;
a light attached to the microprocessor;
a speaker attached to the microprocessor;
a battery attached to the microprocessor.

7. The apnea monitor of claim 6, wherein the apnea monitor is disposable.

8. The apnea monitor of claim 6, wherein the light illuminates upon the detection of an apneic event.

9. The apnea monitor of claim 6, further comprising a vibration unit attached to the microprocessor.

10. The apnea monitor of claim 9, wherein the adhesive element is circular.

11. The apnea monitor of claim 6, wherein the microprocessor further comprises software for audio analysis.

12. The apnea monitor of claim 11, further comprising a reset button attached to the microprocessor.

13. The apnea monitor of claim 12, further comprising a light source to indicate power.

14. The apnea monitor of claim 13, wherein the microprocessor further comprises wireless communication hardware.
